(19) United States
(12) Patent Application Publication
(10) Pub. No.: US 2025/0352247 A1
(43) Pub. Date: Nov. 20, 2025

(54) FRACTURE REDUCTION MECHANISM FOR PELVIC FRACTURE MINIMALLY INVASIVE SURGERY

(71) Applicants: THE FIRST MEDICAL CENTER CHINESE PLA GENERAL HOSPITAL, Beijing (CN); UNIV BEIJING TECHNOLOGY, Beijing (CN)

(72) Inventors: Peifu TANG, Beijing City (CN); Jianfeng Li, Beijing City (CN); Jingxin Zhao, Beijing City (CN); Mingjie Dong, Beijing City (CN); Hua Chen, Beijing City (CN); Qinglong Lun, Beijing City (CN)

(21) Appl. No.: 19/222,202

(22) Filed: May 29, 2025

Related U.S. Application Data
(63) Continuation of application No. PCT/CN2022/141322, filed on Dec. 23, 2022.

(30) Foreign Application Priority Data
Nov. 29, 2022 (CN) 202211506151.5

(51) Int. Cl. A61B 17/64; A61B 17/00

(52) U.S. Cl. CPC A61B 17/6433; A61B 17/6408; A61B 17/6416; A61B 2017/00398

(57) ABSTRACT

A fracture reduction mechanism for minimally invasive pelvic fracture surgery, comprising a support assembly, a drive assembly, a self-rotating clamping assembly, and a healthy side fixation assembly, wherein the drive assembly comprises a synchronous rotation module (21), a first ball screw module (22), a second ball screw module (23), a third ball screw module (24), and an arc guide rail module (33); the mechanism forms a frame-like closed structure through connection with an operating table (1) via bedside rails (2) on both sides of the operating table (1), the support assembly and healthy side fixation assembly are slidably connected with the bedside rails (2). By manually adjusting the linear movement distances and fixed-axis rotation angles of the above mechanism, six degrees of freedom translation and rotation of the pelvic fracture block can be achieved. The mechanism features high rigidity and load-bearing capacity, good portability, simple assembly and operation, high motion precision, and its motion center is a virtual point in space, the position of which can be adjusted according to patient position and fracture type, providing convenience for surgery.

### TECHNICAL FIELD

The present application relates to the field of medical equipment technology, specifically to a fracture reduction mechanism for minimally invasive pelvic fracture surgery.

### BACKGROUND

With the rapid development of modern society, the incidence of pelvic fractures has been increasing yearly. Pelvic fractures have high disability rates (37%) and mortality rates (30-60%). Traditional surgery involves significant trauma, which critically ill patients cannot tolerate. While minimally invasive pelvic fracture surgery offers advantages such as minimal trauma and quick recovery, fracture reduction remains challenging, and complications such as nerve damage and limb dysfunction are related to poor fracture reduction.

Pelvic fracture reduction requires significant force, and due to factors such as soft tissue incarceration and fracture end interlocking, fracture displacement cannot be directly reduced through lower limb traction. It often requires initial horizontal traction of the pelvic fracture block to unlock the fracture ends. The entire reduction process typically requires forces of 400-500N or even higher, and must be performed under intraoperative fluoroscopy, posing significant health risks to medical staff.

In recent years, reports of fracture surgery robots have gradually increased, mainly used for limb fractures, with configurations primarily divided into serial and parallel mechanisms. The former mainly uses robotic arm-type robots, while the latter mainly uses Ilizarov external frames or Stewart platforms. Serial mechanisms have a large range of motion but low rigidity and load capacity, and errors are amplified through multiple joints. There are reports of using UR16e robots for pelvic fracture reduction, but their effective payload of only 160N cannot meet clinical requirements. Parallel mechanisms have high rigidity, load capacity, and precision, but when applied to pelvic fractures, they interfere with surgical operations and intraoperative X-ray imaging. Other research has combined serial and parallel mechanisms, but still primarily uses serial mechanisms, controlling holding screws through parallel mechanisms. These mechanisms are typically placed beside the operating table, and to provide effective payload, the mechanism volume and weight must be very large, resulting in poor portability and unsuitability for situations requiring rapid transfer and transport.

### SUMMARY

The object of the present application is to provide a fracture reduction mechanism for minimally invasive pelvic fracture surgery to solve the problems of large mechanism volume and weight, low load capacity, low rigidity, poor portability, and low precision in related technology.

To achieve the above object, the present application provides the following technical solution:

A fracture reduction mechanism for minimally invasive pelvic fracture surgery, wherein: the pelvic fracture reduction mechanism comprises a support assembly, a drive assembly, a self-rotating clamping assembly, and a healthy side fixation assembly; wherein,
the drive assembly comprises a synchronous rotation module, a first ball screw module, a second ball screw module, a third ball screw module, and an arc guide rail module;
the first ball screw module can drive the self-rotating clamping assembly and the pelvic fracture block held thereby to move horizontally, enabling the fracture ends to separate and unlock, providing movement space for precise reduction of the fracture block; the support assembly is symmetrically arranged on the bedside rails on both sides of the operating table and slidably connected therewith, capable of translating along the bedside rail and in a direction perpendicular to the bed surface, reducing displacement of the fracture ends and implementing rough fracture reduction; the drive assembly drives the self-rotating clamping assembly to perform six degrees of freedom movement, implementing precise fracture reduction;
the support assembly is symmetrically arranged on the bedside rails on both sides of the operating table and slidably connected therewith, the mechanism forms a frame-like closed structure through connection with the operating table via bedside rails on both sides, providing high rigidity and load capacity; through the closed structure, the mechanism can utilize the operating table to provide self-counteracting stress, outputting large effective payload;
the synchronous rotation module is hinged with a U-shaped slot at the upper end of the column of the support assembly;
the first ball screw module is arranged along the short axis direction of the bed, with both ends fixedly connected to the upper end of the upper support arm of the synchronous rotation module;
the arc guide rail module is arranged along the short axis direction of the bed, the arc rack guide rail plane of the arc guide rail module is parallel to the base plane of the first ball screw module and located in front of it, mounted on the sliding platform of the first ball screw module through a bottom plate fixing ring;
the second ball screw module is arranged in front of the arc guide rail module and parallel to the arc rack guide rail plane, connected to the arc guide rail sliding platform of the arc guide rail module through the sliding platform of the second ball screw module; the third ball screw module is arranged below the second ball screw module and perpendicular to the second ball screw module, connected to the lower support plate of the second ball screw module through the sliding platform of the third ball screw module;
the movement of the ball screw module and arc guide rail module is controlled by ball screw nuts and rack and pinion, providing stable transmission, high motion precision, and convenient operation;
the self-rotating clamping assembly is located in front of the third ball screw module and fixedly installed on its front support plate, used for clamping holding screws and driving the fracture block to rotate about a fixed axis;
the rotation axes of the three fixed-axis rotation components including the self-rotating clamping assembly, arc guide rail module, and synchronous rotation module intersect at one point, which is the rotation center of the entire mechanism;
the healthy side fixation assembly is slidably connected with the bedside rail, used for firmly fixing the healthy side hemipelvis;
through the above mechanism, six degrees of freedom translation and rotation of the pelvic fracture block can be achieved.

Further, the support assembly comprises a support column sliding seat, a support column, and a column locking module, the support assembly is located on both sides of the operating table in a symmetric distribution; the support column sliding seat is slidably connected with the bedside rails on both sides of the operating table, driving the mechanism to translate along the bed long axis, and is locked and fixed through the locking handwheel of the support column sliding seat; the support column is vertically installed in the trapezoidal slot of the support column sliding seat, driving the mechanism to rise and fall perpendicular to the bed surface, and is locked and fixed through the column locking module.

Further, the column locking module comprises a fixed rack, a movable rack, a first sliding block, a lead screw, a sliding saddle, and a lead screw support seat, the fixed rack is arranged on both sides of the support column and fixedly connected therewith, the sliding saddle and lead screw support seat are arranged on both sides of the support column, located on the support column sliding seat and fixedly connected therewith, the first sliding block is slidably connected with the sliding saddle, one end of the lead screw is connected to the first sliding block through a nut, and the other end is threadedly connected with the lead screw support seat, the movable rack is fixedly connected with the first sliding block, achieving locking and fixing of the support column's rise and fall through the engagement of the fixed rack and movable rack.

Further, the synchronous rotation module comprises a first worm gear drive, a first angular contact ball bearing, a stepped shaft, a bearing end cap, an upper support arm, a lower support arm, a support rod, a support rod sliding seat, and a support rod hinge seat, the first angular contact ball bearing is fixedly connected with the slot wall of the U-shaped slot at the upper end of the support column, the stepped shaft is supported by two first angular contact ball bearings, one end of the lower support arm is fixedly connected with the stepped shaft, and the other end is fixedly connected with the upper support arm, used for supporting and fixing the first ball screw module; the first worm gear drive and bearing end cap are installed on the outer side of the support column, the drive output shaft is fixedly connected with one end of the stepped shaft, driving the lower support arm to rotate about a fixed axis and lock, the installation positions of the first worm gear drive and bearing end cap can be interchanged according to the doctor's standing position requirements when performing reduction on left and right sides; both ends of the support rod are hinged with the support rod hinge seat and support rod sliding seat respectively, used for auxiliary support and locking of fixed-axis rotation, the support rod hinge seat is fixedly connected with the upper support arm, the support rod sliding seat is slidably connected with the healthy side bedside rail, and is locked and fixed through the locking handwheel of the support rod sliding seat.

Further, the ball screw module adopts a ball screw and double-sided sliding saddle guide rail structure, both ends of the ball screw are supported by third angular contact ball bearings, and the module end face is equipped with a second handwheel and a clamp. Wherein, the first ball screw module drives the arc guide rail module to translate along the bed short axis direction, both ends are equipped with second handwheels and clamps to meet the requirements of left and right side reduction; the second ball screw module drives the self-rotating clamping assembly to move along the radial direction of the arc rack guide rail; the third ball screw module drives the self-rotating clamping assembly to move along the axial direction of the arc rack guide rail.

Further, the arc guide rail module comprises a bottom plate fixing ring, an arc bottom plate, an arc rack guide rail, an arc guide rail sliding platform, guide wheels, support blocks, a second worm gear drive, and a gear, the arc bottom plate is fixedly installed on the front side of the bottom plate fixing ring, the arc rack guide rail is fixedly installed on the arc bottom plate; the guide wheels and support blocks are installed on the arc guide rail sliding platform, connected with the arc rack guide rail through rolling connection and sliding connection respectively, used for fixing and guiding the arc guide rail sliding platform; the second worm gear drive is fixedly connected with the arc guide rail sliding platform, the gear is fixedly installed on the worm wheel shaft of the second worm gear drive, and meshes with the arc rack, driving the arc guide rail sliding platform to move along the arc rack guide rail.

Further, the worm gear drive comprises a housing, second angular contact ball bearings, a worm wheel, a worm wheel shaft, a worm shaft and a first handwheel, the second angular contact ball bearings are fixedly connected with the housing support wall, the worm wheel shaft and worm shaft are supported by two second angular contact ball bearings respectively, the worm wheel is fixedly installed on the worm wheel shaft, and meshes with the worm shaft, the handwheel is fixedly connected with the worm shaft, driving the worm wheel shaft to rotate and output power.

Further, the self-rotating clamping assembly comprises a bottom plate, support seats, fourth angular contact ball bearings, a secondary pin rotation sleeve, a secondary pin guide rod, a secondary pin bracket, a main pin clamp, a secondary pin clamp, quick-locking bolts, a clamp, a third handwheel, a main pin and a secondary pin, the bottom plate is fixedly connected with the front support plate of the third ball screw module, the support seats are fixedly installed on both sides of the bottom plate, wherein the upper support plate can be installed with an external arc caliper for measuring the rotation angle of the pelvic fracture block; the fourth angular contact ball bearings and clamp are fixedly connected with the support seats, the secondary pin rotation sleeve is supported by the fourth angular contact ball bearings; the main pin and secondary pin are holding screws for the pelvic fracture block, the main pin and secondary pin pass through the clamps and are locked and fixed through the elastic collets of the clamps, the main pin clamp is installed in the secondary pin rotation sleeve, the secondary pin clamp is installed in the secondary pin bracket, both are locked and fixed through quick-locking bolts; the main pin axis remains parallel to the second ball screw module axis and aligned with the arc rack guide rail radial direction; the main pin and secondary pin hold the pelvic fracture block, the main pin and secondary pin are inserted approximately orthogonally into the pelvis, with the main pin along the direction from anterior inferior iliac spine to posterior inferior iliac spine, and the secondary pin in the direction of the transverse acetabular screw, the two screws are distributed at close distances, the holding screw structure is compact, providing more precise control of the fracture block; when torque is applied to the secondary pin rotation sleeve, it drives the main pin, secondary pin and fracture block to synchronously rotate about the main pin axis to prevent the main pin from slipping within the fracture block, which would cause holding and fixation failure; the secondary pin bracket is slidably connected with the secondary pin guide rod, the secondary pin bracket can extend and retract to adapt to different patients; the clamps, secondary pin rotation sleeve, and secondary pin bracket have through holes, providing some guidance function.

Further, the healthy side fixation assembly comprises a support sliding block, a first support vertical shaft, a sliding shaft sleeve, a support horizontal shaft, a sliding shaft sleeve, a second support vertical shaft, a cross connector, a fixing rod, a pin rod fixing clamp, external fixation pins and a locking handwheel, the support sliding block is slidably connected with the healthy side bedside rail, and is locked and fixed through the locking handwheel; the first support vertical shaft is fixedly connected with the support sliding block, the support horizontal shaft is slidably connected with the first support vertical shaft through the sliding shaft sleeve, the second support vertical shaft is fixedly connected with the support horizontal shaft through the cross connector, the fixing rod is slidably connected with the support horizontal shaft through the pin rod fixing clamp, the external fixation pins are slidably connected with the fixing rod through the pin rod fixing clamp; the sliding shaft sleeve, fixing rod and pin rod fixing clamp adapt to different poses of external fixation pins. The healthy side fixation assembly is set up as two sets.

Further, the values of translation and rotation can be visually displayed, wherein, the translation movement scale precision of the support assembly and ball screw module is set to 1 mm, the scale precision of the fixed-axis rotation external arc caliper and arc rack guide rail is set to 1°; the stroke of the support column is 80 mm˜120 mm, the first ball screw module is set with different strokes to adapt to standard operating tables of different widths, the strokes of the second and third ball screw modules are 80 mm˜120 mm and 40˜80 mm respectively; the stroke of the arc rack guide rail is 110°˜140°, the self-rotation stroke of the self-rotating clamping assembly is 220°˜270°. The above movement and fixed-axis rotation strokes can be adjusted according to actual application requirements.

Compared with prior art, the beneficial effects of the present application are:

The mechanism adopts a frame-like, modular design with easy assembly and disassembly connections between modules, forming a closed rigid structure with the operating table, providing high rigidity and load capacity. Since pelvic fracture reduction first requires horizontal traction of the fracture block, the force required for unlocking fracture ends is very large. Through the closed structure, the mechanism utilizes the operating table to provide self-counteracting stress, enabling the first ball screw module to output large effective payload without relying on a heavy independent body, reducing mechanism volume and weight while improving portability and effective payload.

Existing similar mechanisms all implement fracture reduction through overall mechanism movement, easily leading to accumulation and amplification of reduction errors. To reduce accumulated errors from mechanism movement, this mechanism performs fracture reduction in two steps: rough reduction and precise reduction. Through the rise, fall and translation of the support assembly, the mechanism can implement rough reduction of the fracture block along the bed long axis (Y-axis) and perpendicular to the bed surface (Z-axis) direction, reducing fracture displacement. Based on this, the drive assembly drives the self-rotating clamping assembly to perform precise fracture reduction. The mechanism movement is controlled by ball screw modules and rack and pinion, providing high precision and convenient operation. The worm gear drives of the synchronous rotation module and arc guide rail module have anti-backdriving self-locking and force amplification functions, maintaining real-time position of the fracture block and reducing doctor workload. Unlike drive components of similar mechanisms that cannot intuitively read fracture block movement stroke, the stroke of each drive component in this mechanism can be directly read through scales and calipers, providing more intuitive guidance for doctors performing reduction operations.

Similar mechanisms often choose to insert holding screws into the iliac crest and anterior inferior iliac spine, where the iliac crest bone is relatively weak with risk of refracture; meanwhile, the two screws are far apart, requiring large space for gripping tools, unfavorable for precise operation. To avoid the risk of refracture when iliac crest screws are under high stress, the main pin and secondary pin of this mechanism's holding screws are inserted approximately orthogonally into the hardest part of the pelvis-above the acetabulum, providing firm grip of the fracture block. The two screws are close together, making the holding structure compact and providing more precise control of the fracture block.

The main components of this mechanism do not obstruct the pelvis, reserving sufficient space for taking pelvic anteroposterior radiographs (as shown in FIG. 11) and inlet view radiographs (as shown in FIG. 12), meeting the requirements for fracture displacement measurement and intraoperative navigation. The mechanism has a simple structure, convenient assembly and disassembly, easy sterilization and transportation, can shorten operation time, reduce intraoperative fluoroscopy, lower surgical difficulty, and provide reliable guarantee for doctors performing minimally invasive fixation after fracture reduction.

### BRIEF DESCRIPTION OF THE DRAWINGS

FIG. 1 is an axonometric view of the fracture reduction mechanism for minimally invasive pelvic fracture surgery of the present application;

FIG. 2 is a front view of the fracture reduction mechanism for minimally invasive pelvic fracture surgery of the present application;

FIG. 3 is a right view of the fracture reduction mechanism for minimally invasive pelvic fracture surgery of the present application;

FIG. 4 is a structural schematic diagram of the support assembly;

FIG. 5 is a structural schematic diagram of the synchronous rotation module;

FIG. 6 is a structural schematic diagram of the arc guide rail module;

FIG. 7 is a schematic diagram of the first ball screw module;

FIG. 8 is a structural schematic diagram of the self-rotating clamping assembly;

FIG. 9 is a structural schematic diagram of the healthy side fixation assembly;

FIG. 10 is a simplified mechanism diagram showing the working principle of the fracture reduction mechanism for minimally invasive pelvic fracture surgery of the present application;

FIG. 11 is a schematic diagram of pelvic anteroposterior radiograph;

FIG. 12 is a schematic diagram of pelvic inlet view radiograph.

### DESCRIPTION OF REFERENCE NUMERALS

operating table 1, bedside rail 2, support column sliding seat 3, column locking module 4, support column 5, fixed rack 6, movable rack 7, first sliding block 8, lead screw 9, sliding saddle 10, lead screw support seat 11, first worm gear drive 12, first angular contact ball bearing 13, stepped shaft 14, bearing end cap 15, upper support arm 16, lower support arm 17, support rod 18, support rod sliding seat 19, support rod hinge seat 20; synchronous rotation module 21, first ball screw module 22, second ball screw module 23, third ball screw module 24; bottom plate fixing ring 25, arc bottom plate 26, arc rack guide rail 27, arc guide rail sliding platform 28, guide wheel 29, support block 30, second worm gear drive 31, gear 32, arc guide rail module 33; housing 34, second angular contact ball bearing 35, worm wheel 36, worm wheel shaft 37, worm shaft 38, first handwheel 39, second worm wheel shaft 40; ball screw 41, base 42, sliding saddle 43, support plate 44, third angular contact ball bearing 45, ball screw nut 46, nut sleeve 47, second sliding block 48, sliding platform plate 49, clamp 50, second handwheel 51; bottom plate 52, support seat 53, fourth angular contact ball bearing 54, secondary pin rotation sleeve 55, secondary pin guide rod 56, secondary pin bracket 57, main pin clamp 58, secondary pin clamp 59, quick-locking bolt 60, clamp 61, third handwheel 62; elastic collet 63, locking nut 64, screw sleeve 65; support sliding block 66, first support vertical shaft 67, sliding shaft sleeve 68, short support horizontal shaft 69, long support horizontal shaft 70, second support vertical shaft 71, cross connector 72, fixing rod 73, pin rod fixing clamp 74, locking handwheel 75; upper clamp 76, lower clamp 77, bolt 78, spring 79.

### DETAILED DESCRIPTION

The following is a detailed description of the present application in conjunction with the embodiments shown in the drawings. However, it should be noted that these embodiments are not limitations on the present application, and any functional, methodological, or structural equivalents or substitutions made by those skilled in the art based on these embodiments fall within the scope of protection of the present application.

It should be noted that the terms “first,” “second,” etc. in the specification, claims, and above drawings of this application are used to distinguish similar objects and are not necessarily used to describe a specific order or sequence. It should be understood that such terms can be interchanged under appropriate circumstances to facilitate the description of the embodiments of this application.

In this application, terms such as “upper,” “lower,” “front,” “back,” etc. indicate directional or positional relationships based on the directional or positional relationships shown in the drawings. These terms are mainly used to better describe the present application and its embodiments, and are not intended to limit the indicated devices, elements, or components to have specific orientations or to be constructed and operated in specific orientations. Moreover, some of these terms can be used to express other meanings besides indicating directional or positional relationships, for example, the term “upper” in some cases may also be used to indicate a dependent or connected relationship. Those skilled in the art can understand the specific meanings of these terms in this application according to specific circumstances. Additionally, terms such as “set,” “equipped with,” “fixed,” etc. should be broadly interpreted, and the term “fixedly connected” refers to detachable connection through bolts. Those skilled in the art can understand the specific meanings of the above terms in this application according to specific circumstances.

To improve the precision of minimally invasive pelvic fracture surgery, reduce surgical difficulty, and reduce intraoperative radiation, surgical robots are used for fracture reduction in related technology. Since the force required for pelvic fracture reduction is very large, existing robot configurations used for pelvic fracture reduction need to rely on heavy cabinets or platforms standing beside the operating table to provide sufficient power. However, in some cases, the robot needs to be quickly transported, thus placing high requirements on the robot's volume, weight, and precision.

To this end, this application provides a pelvic fracture reduction mechanism to achieve the purpose of having greater output force, sufficient workspace, and higher precision while maintaining good portability of the mechanism. Specifically, as shown below:

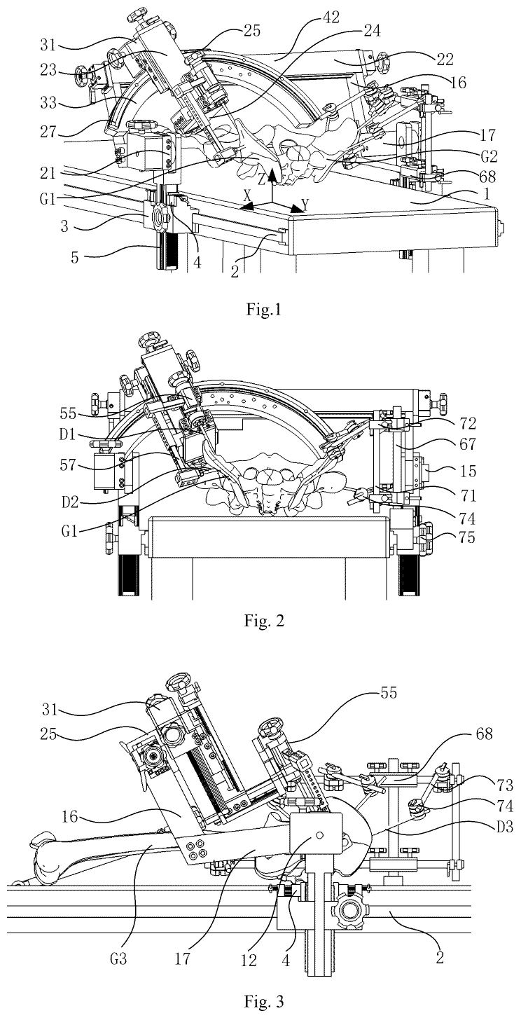

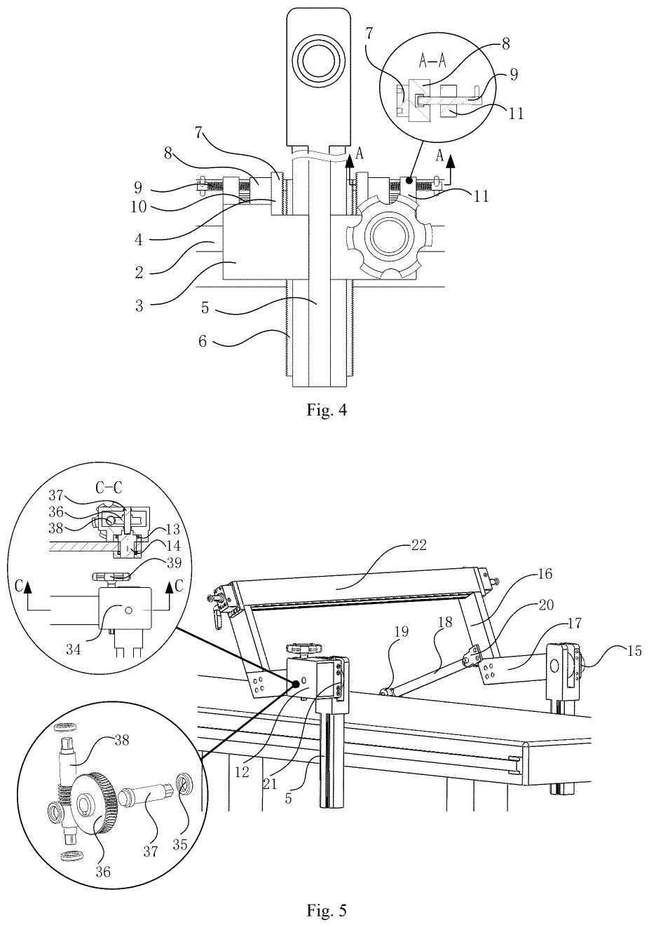

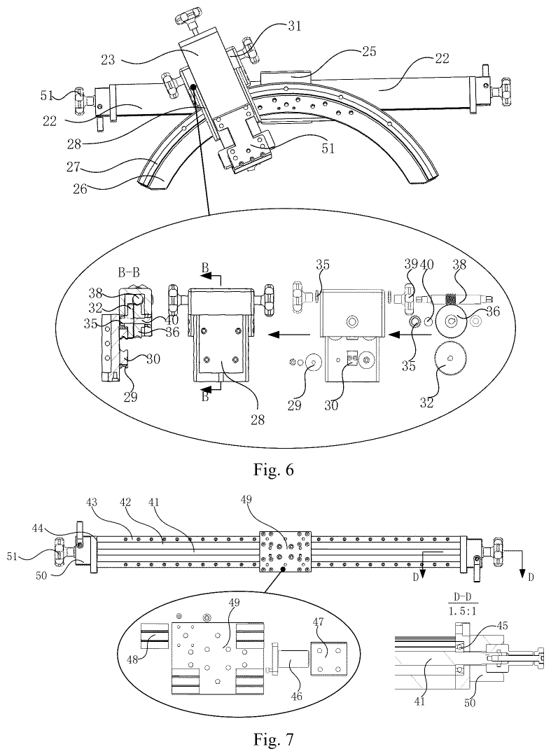

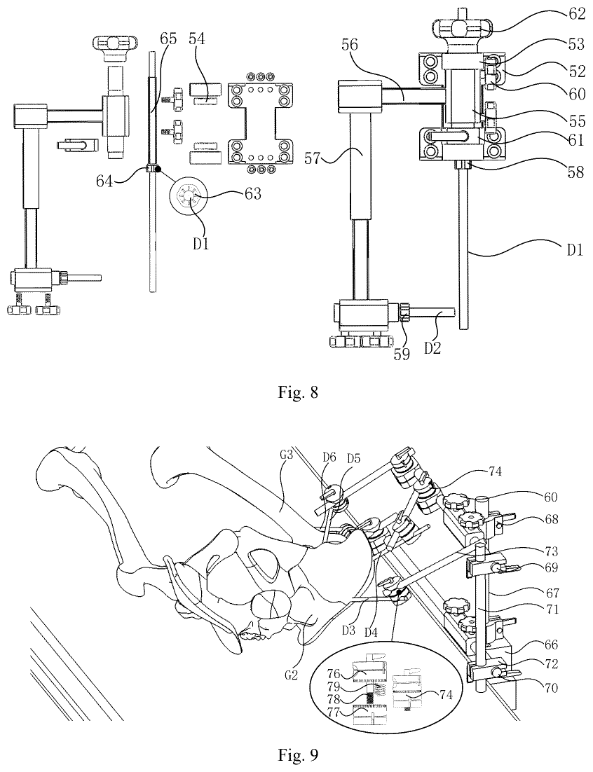

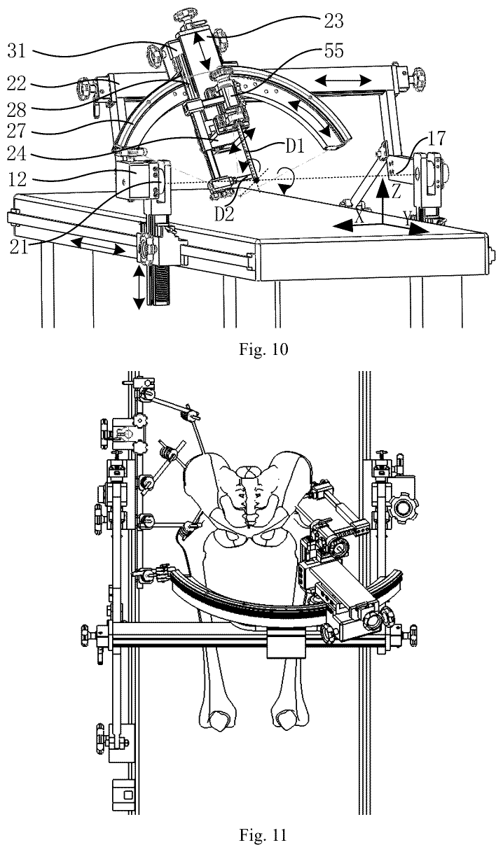

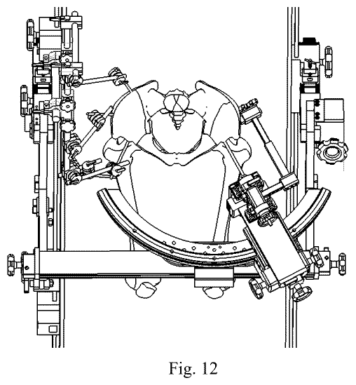

Referring to FIGS. 1 to 12, FIG. 1 is an axonometric view of the fracture reduction mechanism for minimally invasive pelvic fracture surgery of the present application, FIG. 2 is a front view of the fracture reduction mechanism for minimally invasive pelvic fracture surgery of the present application, FIG. 3 is a right view of the fracture reduction mechanism for minimally invasive pelvic fracture surgery of the present application, FIG. 4 is a structural schematic diagram of the support assembly, FIG. 5 is a structural schematic diagram of the synchronous rotation module, FIG. 6 is a structural schematic diagram of the arc guide rail module, FIG. 7 is a schematic diagram of the first ball screw module, FIG. 8 is a structural schematic diagram of the self-rotating clamping assembly, FIG. 9 is a structural schematic diagram of the healthy side fixation assembly, FIG. 10 is a simplified mechanism diagram showing the working principle of the fracture reduction mechanism for minimally invasive pelvic fracture surgery of the present application, FIG. 11 is a schematic diagram of pelvic anteroposterior radiograph, FIG. 12 is a schematic diagram of pelvic inlet view radiograph.

In this embodiment, a fracture reduction mechanism for minimally invasive pelvic fracture surgery is provided. As shown in FIGS. 1-10, the fracture reduction mechanism comprises a support assembly, a drive assembly, a self-rotating clamping assembly, and a healthy side fixation assembly; wherein, the drive assembly comprises a synchronous rotation module 21, a first ball screw module 22, a second ball screw module 23, a third ball screw module 24, and an arc guide rail module 33; the support assembly is symmetrically arranged on the bedside rails 2 on both sides of the operating table 1 and slidably connected therewith, the mechanism forms a closed structure through connection with the operating table 1 via bedside rails 2 on both sides; the synchronous rotation module 21 is hinged with a U-shaped slot at the upper end of the column 5 of the support assembly; the first ball screw module 22 is arranged along the bed short axis (X-axis) direction, with both ends fixedly connected to the upper end of the upper support arm 16 of the synchronous rotation module 21; the arc guide rail module 33 is arranged along the bed short axis (X-axis) direction, the arc rack guide rail 27 plane is parallel to the base 42 plane of the first ball screw module 22 and located in front of it, mounted on the sliding platform of the first ball screw module 22 through a bottom plate fixing ring 25; the second ball screw module 23 is arranged in front of the arc rack guide rail 27 and parallel to its plane, connected to the sliding platform of the arc guide rail module 33 through its sliding platform; the third ball screw module 24 is arranged below the second ball screw module 23 and perpendicular to the second ball screw module 23, connected to the lower support plate of the second ball screw module 23 through its sliding platform; the self-rotating clamping assembly is located in front of the third ball screw module 24 and fixedly installed on its front support plate, used for clamping holding screws D1, D2 and driving the pelvic fracture block to rotate about a fixed axis; the healthy side fixation assembly is slidably connected with the bedside rail 2, used for firmly fixing the healthy side hemipelvis G2; through the above mechanism, six degrees of freedom translation and rotation of the pelvic fracture block can be achieved.

In this embodiment, as shown in FIGS. 1, 2, and 4, the support assembly comprises a support column sliding seat 3, a column locking module 4, and a support column 5, the support assembly is located on both sides of the operating table 1 in a symmetric distribution; the support column sliding seat 3 is slidably connected with the bedside rails 2 on both sides of the operating table 1, through implementing lower limb traction on the affected side or simultaneously applying pushing force to both sides of the mechanism to push the sliding platform 3, driving the mechanism to translate along the bed long axis (Y-axis), and is locked and fixed through the locking handwheel of the support column sliding seat 3; the support column 5 has a trapezoidal sliding track vertically installed in the trapezoidal sliding slot of the support column sliding seat 3, driving the mechanism to rise and fall perpendicular to the bed surface (Z-axis), and is locked and fixed through the column locking module 4 on both sides of the support column 5; the support assembly can rise and fall as a whole to adapt to different fracture types, and after installation of the mechanism, can drive the mechanism to implement rough reduction of the pelvic fracture block along the bed long axis (Y-axis) or perpendicular to the bed surface (Z-axis) direction;

The column locking module 4 comprises a fixed rack 6, a movable rack 7, a first sliding block 8, a lead screw 9, a sliding saddle 10, and a lead screw support seat 11, the sliding saddle 10 and lead screw support seat 11 are arranged on both sides of the support column 5, located on the support column sliding seat 3 and fixedly connected therewith, the first sliding block 8 is slidably connected with the sliding saddle 10, one end of the lead screw 9 is connected to the first sliding block 8 through a nut, and the other end is threadedly connected with the lead screw support seat 11, the movable rack 7 is fixedly connected with the first sliding block 8, the fixed rack 6 is fixedly connected with the support column 5, simultaneously raising or lowering the support column 5 on both sides of the operating table 1, through rotating the lead screw 9 to push the first sliding block 8, driving the movable rack 7 to mesh with the fixed rack 6, achieving locking and fixing of the support column 5′s rise and fall.

In this embodiment, as shown in FIGS. 1, 3, and 5, the synchronous rotation module 21 comprises a first worm gear drive 12, two first angular contact ball bearings 13, a stepped shaft 14, a bearing end cap 15, an upper support arm 16, a lower support arm 17, a support rod 18, a support rod sliding seat 19, and a support rod hinge seat 20; the two first angular contact ball bearings 13 are fixedly installed in the slot walls of the U-shaped slot at the upper end of the support column 5, the stepped shaft 14 is supported by two first angular contact ball bearings 13, the stepped shaft 14 is fixedly connected with the lower support arm 17; the first worm gear drive 12 and bearing end cap 15 are installed on the outer side of the support column 5 through quick-release bolts, the output shaft of the first worm gear drive 12 is fixedly connected with one end of the stepped shaft 14, driving the lower support arm 17 to rotate about a fixed axis and lock, the installation positions of the first worm gear drive 12 and bearing end cap 15 can be interchanged according to the doctor's standing position requirements when performing reduction on left and right sides; the upper support arm 16 is fixedly connected with the lower support arm 17, used for supporting and fixing the first ball screw module 22; both ends of the support rod 18 are hinged with the support rod hinge seat 20 and support rod sliding seat 19 respectively, used for auxiliary support and locking of fixed-axis rotation, the support rod hinge seat 20 is fixedly connected with the upper support arm 16, the support rod sliding seat 19 is slidably connected with the healthy side bedside rail 2, and is locked and fixed through the locking handwheel of the support rod sliding seat 19.

In this embodiment, as shown in FIGS. 1 and 7, the first ball screw module 22 comprises a ball screw 41, a base 42, two sliding saddles 43, two support plates 44, two third angular contact ball bearings 45, a ball screw nut 46, a nut sleeve 47, four second sliding blocks 48, a sliding platform plate 49, a clamp 50, and a second handwheel 51, the support plates 44 equipped with third angular contact ball bearings 45 are fixed at both ends of the base 42, both ends of the ball screw 41 are supported by two third angular contact ball bearings 45 and fitted with a ball screw nut 46, the ball screw nut 46 is fitted with a nut sleeve 47, the upper end of the nut sleeve 47 is equipped with a sliding platform plate 49, the bottom surface of the sliding platform plate 49 is equipped with second sliding blocks 48, and is slidably connected with the sliding saddles 43 through their sliding grooves, the module end face is equipped with a second handwheel 51 and a clamp 50, used for driving the ball screw nut 46 to move along the ball screw 41 and lock. The three ball screw modules 22, 23, 24 have the same structure and connection method but different strokes, all adopting ball screw and double-sided sliding saddle guide rail structure, without repeated description. Among them, both ends of the first ball screw module are equipped with second handwheels 51 and clamps 50 to meet the requirements of left and right side reduction.

In this embodiment, as shown in FIGS. 1 and 6, the arc guide rail module 33 comprises a bottom plate fixing ring 25, an arc bottom plate 26, an arc rack guide rail 27, an arc guide rail sliding platform 28, four guide wheels 29, two support blocks 30, a second worm gear drive 31 and a gear 32, the arc bottom plate 26 is fixedly installed on the front side of the bottom plate fixing ring 25, the arc rack guide rail 27 is fixedly connected with the arc bottom plate 26, the arc rack guide rail 27 moves linearly along the bed short axis (X-axis) through the sliding platform of the first ball screw module 22; the four guide wheels 29 and two support blocks 30 are installed on the arc guide rail sliding platform 28, connected with the arc rack guide rail 27 through rolling connection and sliding connection respectively, the second worm gear drive 31 is fixedly connected with the arc guide rail sliding platform 28, the gear 32 is fixedly installed on the second worm wheel shaft 40 of the second worm gear drive 31, and meshes with the rack of the arc rack guide rail 27, rotating the first handwheel 39 drives the arc guide rail sliding platform 28 to move along the arc rack guide rail 27.

In this embodiment, as shown in FIGS. 1, 5, and 6, the worm gear drive 12 comprises a housing 34, four second angular contact ball bearings 35, a worm wheel 36, a first worm wheel shaft 37, a worm shaft 38, and a first handwheel 39, the four second angular contact ball bearings 35 are embedded in the four support walls of the housing 34, the first worm wheel shaft 37 and worm shaft 38 are supported by two second angular contact ball bearings 35 respectively, the worm wheel 36 is fixedly installed on the first worm wheel shaft 37 and meshes with the worm shaft 38, the first handwheel 39 is fixedly connected with the worm shaft 38, driving the first worm wheel shaft 37 to rotate and output power; the second worm gear drive 31 has a different housing 34 structure from the first worm gear drive 12, the second worm wheel shaft 40 is a cantilever shaft with gear 32 and worm wheel 36 fixed on it, one end is connected to the housing support plate through second angular contact ball bearings 35, the other end is equipped with a shaft end fixer 33, the remaining structure and connection method are the same as the first worm gear drive 12, without repeated description.

In this embodiment, as shown in FIGS. 1, 2, and 8, the self-rotating clamping assembly comprises a bottom plate 52, two support seats 53, two fourth angular contact ball bearings 54, a secondary pin rotation sleeve 55, a secondary pin guide rod 56, a secondary pin bracket 57, a main pin clamp 58, a secondary pin clamp 59, quick-locking bolts 60, a clamp 61, a third handwheel 62, a main pin D1 and a secondary pin D2, the bottom plate 52 is fixedly connected with the front support plate of the third ball screw module, the two support seats 53 are fixedly connected with the bottom plate 52, the two fourth angular contact ball bearings 54 are embedded in the two support seats 53, the secondary pin rotation sleeve 55 is supported by two fourth angular contact ball bearings 54, the main pin clamp 58 passes through the secondary pin rotation sleeve 55 and is fixedly connected with it through quick-locking bolts 60, the secondary pin bracket 57 is slidably connected with the secondary pin guide rod 56, the secondary pin clamp 59 is fixedly connected with the secondary pin bracket 57 through quick-locking bolts 60, the clamps 58, 59 consist of elastic collets 63, locking nuts 64, and screw sleeves 65, tightening the locking nuts 64 causes the elastic collets 63 to contract, firmly clamping the screws D1, D2, the main pin DI axis remains parallel to the second ball screw module 23 axis and aligned with the arc rack guide rail radial direction; the secondary pin bracket 57 can extend and retract and is positioned and fixed through bolts to adapt to different patients; the clamp 61 is fitted on the outside of the secondary pin rotation sleeve 55 and fixedly connected with the lower support seat 53, rotating the third handwheel 62 on the upper end of the secondary pin rotation sleeve 55 drives the main pin D1 and secondary pin D2 to rotate around the axis of main pin D1, and is locked and fixed through the clamp 61. The main pin clamp 58, secondary pin clamp 59, secondary pin rotation sleeve 55, and secondary pin bracket 57 have through holes, providing some guidance function.

In this embodiment, as shown in FIGS. 1, 2, 3, and 9, the healthy side fixation assembly comprises a support sliding block 66, a first support vertical shaft 67, two sliding shaft sleeves 68, a short support horizontal shaft 69, a long support horizontal shaft 70, a second support vertical shaft 71, two cross connectors 72, four fixing rods 73, eight pin rod fixing clamps 74, four external fixation pins D3˜D6 and a locking handwheel 75, the support sliding block 66 is slidably connected with the healthy side bedside rail 2, and is locked and fixed through the locking handwheel 75; the first support vertical shaft 67 is fixedly connected with the support sliding block 66, the two support horizontal shafts 69, 70 are slidably connected with the first support vertical shaft 67 through two sliding shaft sleeves 68 and locked and fixed through two quick-locking bolts 60, the sliding shaft sleeves 68 can translate up and down along the first support vertical shaft 67 and lock; the second support vertical shaft 71 is slidably connected and locked with the two support horizontal shafts 69, 70 through two cross connectors 72, the four fixing rods 73 are slidably connected and locked with the support horizontal shafts 69, 70 through four pin rod fixing clamps 74, with three connected to the short support horizontal shaft 69 and one connected to the long support horizontal shaft 70; the pin rod fixing clamps 74 consist of upper clamps 76 with tooth discs and lower clamps 77 connected through bolts 78, the bolts 78 are fitted with springs 79 providing clamping cushioning, the two clamps 76, 77 can rotate around the bolts 78 and are locked and fixed through end tooth disc engagement; the external fixation pins D3˜D5 are fixed with the healthy side hemipelvis G2, D6 is fixed with the healthy side femur G3, and are slidably connected and locked with the four fixing rods 73 through four pin rod fixing clamps 74, used for firmly fixing the healthy side hemipelvis G2.

The working principle of the present application is:

First, the healthy side hemipelvis G2 is firmly fixed to the bedside rail 2 of the operating table 1 through the healthy side fixation assembly. Second, assemble the self-rotating clamping assembly: fixedly connect the holding screws D1, D2 with the self-rotating clamping assembly, as shown in FIGS. 2 and 8, detailed explanation of the self-rotating clamping assembly: place the main pin D1 and secondary pin D2 in the pelvic fracture block G1 respectively, with the main pin D1 along the direction from anterior inferior iliac spine to posterior inferior iliac spine, and the secondary pin D2 in the direction of the transverse acetabular screw, fit the holding screw clamps 58, 59 over the main pin D1 and secondary pin D2 respectively and fix them by rotating the locking nuts 64; fit the secondary pin rotation sleeve 55 over the main pin clamp 58, fit the lower end of the secondary pin bracket 57 over the secondary pin clamp 59, both fixedly connected through quick-locking bolts 60, fit the upper end of the secondary pin bracket 57 over the secondary pin guide rod 56, connect the secondary pin rotation sleeve 55 with the support seat 53 equipped with fourth angular contact ball bearings 54, the support seat 53 is fixedly connected with the bottom plate 52. Finally, assemble the main motion mechanism: install the support assembly on the bedside rails 2 on both sides of the operating table 1 through the support column sliding seat 3, hinge the lower support arm 17 of the synchronous rotation module 21 with the U-shaped slot in the upper part of the support column 5 of the support assembly, fixedly connect the first ball screw module 22 with the upper support arm 16 of the synchronous rotation module 21, mount the arc guide rail module 33 on the sliding platform of the first ball screw module 22 through the bottom plate fixing ring 25; connect the second ball screw module 23 to the arc guide rail sliding platform 28 through its sliding platform; connect the third ball screw module 24 to the lower support plate of the second ball screw module 23 through its sliding platform; fixedly connect the support seat bottom plate 52 of the self-rotating clamping assembly with the support plate of the third ball screw module 24, completing the installation of the fracture reduction mechanism.

As shown in FIGS. 1˜7, 10, 11, and 12, detailed explanation of the mechanism's rotation and translation functions:

The rotation functions of the mechanism are accomplished through the synchronous rotation module, arc guide rail module, and self-rotating clamping assembly. After fixedly connecting the self-rotating clamping assembly with holding screws D1, D2, lock the first, second, and third ball screw modules 22, 23, 24, the second worm gear drive 31 drives the arc guide rail sliding platform 28 to move along the arc rack guide rail 27, driving the pelvic fracture block G1 to rotate around the axis of the arc rack guide rail 27, after rotating to the specified angle, due to the anti-backdriving self-locking characteristic of worm gear transmission, it can lock the arc guide rail sliding platform 28, maintaining the real-time position of the fracture block. The first worm gear drive 12 drives the lower support arm 17 to rotate about a fixed axis, driving the pelvic fracture block G1 to rotate around the line connecting the rotation centers of the synchronous rotation modules 21 on both sides of the operating table, completing rotation around the bed short axis, after rotating to the specified angle, the worm gear transmission self-locks. Unlock the clamp 61 of the self-rotating clamping assembly, rotate the third handwheel 62 to drive the secondary pin rotation sleeve 55 to rotate around the main pin DI axis, driving the pelvic fracture block G1 to complete self-rotation around the main pin D1 axis, then lock and fix through the clamp 61. Among them, both the rotation around the bed short axis and self-rotation around the main pin DI axis can be recorded for movement stroke and rotation degrees through external calipers, while the scale on the arc guide rail can directly display the rotation degrees of the pelvic fracture block G1 around the arc rack guide rail 27 axis. The intersection point of the rotation axis of the arc guide rail sliding platform 28 and the main pin D1 axis is the rotation center of the entire mechanism, this center is located on the line connecting the rotation centers of the synchronous rotation modules 21 on both sides of the bed, as shown by the dotted line in FIG. 10.

The translation functions of the mechanism are divided into overall mechanism translation and precise translation of holding screws, used for rough reduction and precise reduction of the pelvic fracture block G1 respectively. The overall mechanism translation is performed through the support assemblies on both sides along the bed long axis (Y-axis) and perpendicular to the bed surface (Z-axis) direction. The precise translation of holding screws is accomplished by three ball screw modules 22, 23, 24. Among them, the sliding platform of the first ball screw module 22 moves along its axis direction, driving the pelvic fracture block G1 to translate along the bed short axis (X-axis) direction. For the second and third ball screw modules 23, 24, their sliding platforms are fixed while their bases move along the ball screw axis direction, the second ball screw module 23 drives the pelvic fracture block G1 to move along the main pin D1 axis direction, the third ball screw module 24 drives the pelvic fracture block G1 to move along the arc rack guide rail 27 axis direction.

The stroke of each drive component of the mechanism can be directly read through scales and calipers, with values of translation and rotation visually displayed, wherein the translation movement scale precision of the support assembly and ball screw module is set to 1 mm, the scale precision of the fixed-axis rotation external arc caliper and arc rack guide rail is set to 1°; the stroke of the support column is 80 mm˜120 mm, the first ball screw module is set with different strokes to adapt to standard operating tables of different widths, the strokes of the second and third ball screw modules are 80 mm˜120 mm and 40˜80 mm respectively; the stroke of the arc rack guide rail is 110°˜140°, the self-rotation stroke of the self-rotating clamping assembly around the main pin axis is 220°˜270°.

The mechanism can establish fracture reduction paths according to pelvic fracture type and displacement degree through image registration technology, optical navigation, or intraoperative CT equipment. Finally, fracture reduction is implemented through manual adjustment of mechanism component translation and rotation, first performing overall mechanism translation for rough reduction, then adjusting fracture block posture, and finally adjusting holding screw positions to complete precise fracture reduction.

For unilateral pelvic fractures with cranial displacement (AO/OTA-C1 type), the reduction sequence is as follows:
1) Translation along bed short axis (X-axis): rotate the handwheel of the first ball screw module 22, the sliding platform of the first ball screw module 22 translates along the ball screw axis direction, pulling the fracture block G1 to translate along the bed short axis (X-axis), completing fracture end separation and unlocking;
2) Translation along bed long axis (Y-axis): through affected side lower limb traction and bilateral pushing force, translate the mechanism along the bed long axis (Y-axis), reducing the distance between fracture ends;
3) Translation perpendicular to bed surface (Z-axis): through bilateral lifting of the mechanism, translate the mechanism in the direction perpendicular to the bed surface (Z-axis), reducing the distance between fracture ends;
4) Rotation around bed short axis (X-axis): the first worm gear drive 12 drives the lower support arm 17 to rotate about a fixed axis, causing the pelvic fracture block G1 to rotate around the short axis (X-axis);
5) Rotation around main pin D1 axis: rotate the third handwheel 62 to drive the secondary pin rotation sleeve 55, driving the holding screws D1, D2 to rotate around the main pin D1 axis, causing the pelvic fracture block G1 to self-rotate;
6) Rotation around arc rack guide rail 27 axis: the second worm gear drive 31 drives the arc guide rail sliding platform 28 to move along the arc rack guide rail, causing the pelvic fracture block G1 to rotate around the arc rack guide rail 27 axis;
7) Translation along arc rack guide rail 27 axis: rotate the handwheel of the third ball screw module 24, the base of module 24 translates along the ball screw direction, driving the pelvic fracture block G1 to translate along the arc rack guide rail 27 axis direction;
8) Translation along main pin D1 axis direction: rotate the handwheel of the second ball screw module 23, the base of module 23 translates along the ball screw direction, driving the pelvic fracture block G1 to translate along the main pin D1 axis direction;
9) Translation along bed short axis (X-axis): rotate the handwheel of the first ball screw module 22, the sliding block of module 22 moves along the ball screw direction, driving the pelvic fracture block G1 to translate along the bed short axis (X-axis) direction, completing fracture reduction.

For bilateral pelvic fractures, first temporarily fix the side with less displacement, securing it to the operating table through the healthy side fixation assembly, then use the mechanism to perform fracture reduction on the side with greater displacement, then fix that side to the operating table using another set of healthy side fixation assembly, and finally remove the temporary fixation from the side with less displacement and implement fracture reduction using the mechanism. For other types of fractures, the reduction sequence can be adjusted according to actual circumstances.

The series of detailed descriptions listed above are merely specific explanations for feasible implementations of the present application, and they are not intended to limit the scope of protection of the present application. Any equivalent implementation methods or modifications that do not deviate from the technical spirit of the present application should be included within the scope of protection of the present application.

For those skilled in the art, it is obvious that the present application is not limited to the details of the above exemplary embodiments, and can be implemented in other specific forms without departing from the spirit or basic characteristics of the present application. Therefore, from any point of view, the embodiments should be considered as exemplary and non-limiting, the scope of the present application is defined by the appended claims rather than the above description, thus it is intended to encompass all changes falling within the meaning and scope of equivalent elements of the claims within the present application.

1. A fracture reduction mechanism for minimally invasive pelvic fracture surgery, wherein:
the fracture reduction mechanism comprises a support assembly, a drive assembly, a self-rotating clamping assembly, and a healthy side fixation assembly; wherein,
the drive assembly comprises a synchronous rotation module, a first ball screw module, a second ball screw module, a third ball screw module, and an arc guide rail module;
the first ball screw module can drive the self-rotating clamping assembly and the pelvic fracture block held thereby to move horizontally, enabling the fracture ends to separate and unlock, providing movement space for precise reduction of the fracture block; the support assembly is symmetrically arranged on the bedside rails on both sides of the operating table and slidably connected therewith, capable of translating along the bedside rail and in a direction perpendicular to the bed surface, reducing displacement of the fracture ends and implementing rough fracture reduction; the drive assembly drives the self-rotating clamping assembly to perform six degrees of freedom movement, implementing precise fracture reduction;
the fracture reduction mechanism forms a frame-like closed structure through connection with the operating table via the bedside rail, through which the mechanism can utilize the operating table to provide self-counteracting stress;
the synchronous rotation module is hinged with a U-shaped slot at the upper end of the column of the support assembly;
the first ball screw module is arranged along the short axis direction of the bed, with both ends fixedly connected to the upper end of the upper support arm of the synchronous rotation module;
the arc guide rail module is arranged along the short axis direction of the bed, the arc rack guide rail plane of the arc guide rail module is parallel to the base plane of the first ball screw module and located in front of it, mounted on the sliding platform of the first ball screw module through a bottom plate fixing ring;
the second ball screw module is arranged in front of the arc guide rail module and parallel to the arc rack guide rail plane, connected to the arc guide rail sliding platform of the arc guide rail module through the sliding platform of the second ball screw module; the third ball screw module is arranged below the second ball screw module and perpendicular to the second ball screw module, connected to the lower support plate of the second ball screw module through the sliding platform of the third ball screw module;
the movement of the ball screw module and arc guide rail module is controlled by ball screw nuts and rack and pinion;
the self-rotating clamping assembly is located in front of the third ball screw module and fixedly installed on its front support plate, used for clamping the holding screw inserted into the pelvic fracture block, driving the pelvic fracture block to rotate about a fixed axis;
the rotation axes of the three fixed-axis rotation components including the self-rotating clamping assembly, arc guide rail module, and synchronous rotation module intersect at one point, which is the rotation center of the entire mechanism;
by manually adjusting the linear motion distances of the first, second, and third ball screw modules, the pelvic fracture block can be controlled to move linearly along the bed short axis direction, the arc rack guide rail radial direction, and the arc rack guide rail axial direction; by manually adjusting the rotation angles of the synchronous rotation module and arc guide rail module, the fracture block can be controlled to rotate about fixed axes along the bed short axis and arc rack guide rail axis;
through the above mechanism, six degrees of freedom translation and rotation of the pelvic fracture block can be achieved;
the healthy side fixation assembly is slidably connected with the bedside rail, used for fixing the healthy side hemipelvis.

2. The fracture reduction mechanism for minimally invasive pelvic fracture surgery according to claim 1, wherein: the support assembly comprises a support column sliding seat, a support column, and a column locking module, the support assembly is located on both sides of the operating table in a symmetric distribution; the support column sliding seat is slidably connected with the bedside rails on both sides of the operating table, driving the mechanism to translate along the bed long axis, and is locked and fixed through the locking handwheel of the support column sliding seat; the support column is vertically installed in the trapezoidal slot of the support column sliding seat, driving the mechanism to rise and fall perpendicular to the bed surface, and is locked and fixed through the column locking module; the support assembly can rise and fall as a whole to adapt to different fracture types, and after installation of the mechanism, drive the mechanism to implement rough fracture reduction along the bed long axis and perpendicular to the bed surface direction.

3. The fracture reduction mechanism for minimally invasive pelvic fracture surgery according to claim 1, wherein: the column locking module comprises a fixed rack, a movable rack, a first sliding block, a lead screw, a sliding saddle, and a lead screw support seat, the fixed rack is arranged on both sides of the support column and fixedly connected therewith, the sliding saddle and lead screw support seat are arranged on both sides of the support column, located on the support column sliding seat and fixedly connected therewith, the first sliding block is slidably connected with the sliding saddle, one end of the lead screw is connected to the first sliding block through a nut, and the other end is threadedly connected with the lead screw support seat, the movable rack is fixedly connected with the first sliding block, achieving locking and fixing of the support column's rise and fall through the engagement of the fixed rack and movable rack.

4. The fracture reduction mechanism for minimally invasive pelvic fracture surgery according to claim 1, wherein: the synchronous rotation module comprises a first worm gear drive, a first angular contact ball bearing, a stepped shaft, a bearing end cap, an upper support arm, a lower support arm, a support rod, a support rod sliding seat, and a support rod hinge seat, the first angular contact ball bearing is fixedly connected with the slot wall of the U-shaped slot at the upper end of the support column, the stepped shaft is supported by two first angular contact ball bearings, one end of the lower support arm is fixedly connected with the stepped shaft, and the other end is fixedly connected with the upper support arm, used for supporting and fixing the first ball screw module; the first worm gear drive and bearing end cap are installed on the outer side of the support column, the drive output shaft is fixedly connected with one end of the stepped shaft, driving the lower support arm to rotate about a fixed axis and lock, the installation positions of the first worm gear drive and bearing end cap can be interchanged according to the doctor's standing position requirements when performing reduction on left and right sides; both ends of the support rod are hinged with the support rod hinge seat and support rod sliding seat respectively, used for auxiliary support and locking of fixed-axis rotation, the support rod hinge seat is fixedly connected with the upper support arm, the support rod sliding seat is slidably connected with the healthy side bedside rail, and is locked and fixed through the locking handwheel of the support rod sliding seat.

5. The fracture reduction mechanism for minimally invasive pelvic fracture surgery according to claim 1, wherein: the ball screw module adopts a ball screw and double-sided sliding saddle guide rail structure, both ends of the ball screw are supported by third angular contact ball bearings, the module end face is equipped with a second handwheel and a clamp, wherein both ends of the first ball screw module are equipped with second handwheels and clamps to meet the requirements of left and right side reduction.

6. The fracture reduction mechanism for minimally invasive pelvic fracture surgery according to claim 1, wherein: the arc guide rail module comprises a bottom plate fixing ring, an arc bottom plate, an arc rack guide rail, an arc guide rail sliding platform, guide wheels, support blocks, a second worm gear drive, and a gear, the arc bottom plate is fixedly installed on the front side of the bottom plate fixing ring, the arc rack guide rail is fixedly installed on the arc bottom plate; the guide wheels and support blocks are installed on the arc guide rail sliding platform, connected with the arc rack guide rail through rolling connection and sliding connection respectively, used for fixing and guiding the arc guide rail sliding platform; the second worm gear drive is fixedly connected with the arc guide rail sliding platform, the gear is fixedly installed on the worm wheel shaft of the second worm gear drive, and meshes with the arc rack, driving the arc guide rail sliding platform to move along the arc rack guide rail.

7. The fracture reduction mechanism for minimally invasive pelvic fracture surgery according to claim 1, wherein: the worm gear drive comprises a housing, second angular contact ball bearings, a worm wheel, a worm wheel shaft, a worm shaft and a first handwheel, the second angular contact ball bearings are fixedly connected with the housing support wall, the worm wheel shaft and the worm shaft are supported by two second angular contact ball bearings respectively, the worm wheel is fixedly installed on the worm wheel shaft and meshes with the worm shaft, the first handwheel is fixedly connected with the worm shaft, driving the worm wheel shaft to rotate and output power.

8. The fracture reduction mechanism for minimally invasive pelvic fracture surgery according to claim 1, wherein: the self-rotating clamping assembly comprises a bottom plate, support seats, fourth angular contact ball bearings, a secondary pin rotation sleeve, a secondary pin guide rod, a secondary pin bracket, a main pin clamp, a secondary pin clamp, quick-locking bolts, a clamp, a third handwheel, a main pin and a secondary pin, the bottom plate is fixedly connected with the front support plate of the third ball screw module, the support seats are fixedly installed on both sides of the bottom plate, wherein the upper support plate can be installed with an external arc caliper for measuring the rotation angle of the pelvic fracture block around the main pin axis; the fourth angular contact ball bearings and clamp are fixedly connected with the support seats, the secondary pin rotation sleeve is supported by the fourth angular contact ball bearings; the main pin and secondary pin are holding screws for the pelvic fracture block, the main pin and secondary pin pass through the clamps and are locked and fixed through the elastic collets of the clamps, the main pin clamp is installed in the secondary pin rotation sleeve, the secondary pin clamp is installed in the secondary pin bracket, both are locked and fixed through quick-locking bolts; the main pin axis remains parallel to the second ball screw module axis and aligned with the arc rack guide rail radial direction; the main pin and secondary pin are inserted into the pelvic fracture block approximately orthogonally, with the main pin along the direction from anterior inferior iliac spine to posterior inferior iliac spine, and the secondary pin in the direction of the transverse acetabular screw, both screws are placed above the acetabulum; when torque is applied to the secondary pin rotation sleeve, it drives the pelvic fracture block to self-rotate around the main pin axis; combined with the arc guide rail module and synchronous rotation module, three degrees of freedom rotational movement of the fracture block can be implemented; the secondary pin bracket is slidably connected with the secondary pin guide rod, and the secondary pin bracket can extend and retract; the clamps, secondary pin rotation sleeve, and secondary pin bracket have through holes.

9. The fracture reduction mechanism for minimally invasive pelvic fracture surgery according to claim 1, wherein: the healthy side fixation assembly comprises a support sliding block, a first support vertical shaft, a sliding shaft sleeve, a support horizontal shaft, a sliding shaft sleeve, a second support vertical shaft, a cross connector, a fixing rod, a pin rod fixing clamp, external fixation pins and a locking handwheel, the support sliding block is slidably connected with the healthy side bedside rail, and is locked and fixed through the locking handwheel; the first support vertical shaft is fixedly connected with the support sliding block, the support horizontal shaft is slidably connected with the first support vertical shaft through the sliding shaft sleeve, the second support vertical shaft is fixedly connected with the support horizontal shaft through the cross connector, the fixing rod is slidably connected with the support horizontal shaft through the pin rod fixing clamp, the external fixation pins are slidably connected with the fixing rod through the pin rod fixing clamp; the sliding shaft sleeve, fixing rod and pin rod fixing clamp adapt to different poses of external fixation pins; the healthy side fixation assembly is set up as two sets, used for temporary fixation of bilateral pelvis.

10. The fracture reduction mechanism for minimally invasive pelvic fracture surgery according to claim 8, wherein: the stroke of each drive component of the mechanism can be directly read through scales and calipers, the values of translation and rotation can be visually displayed, wherein, the translation movement scale precision of the support assembly and ball screw module is set to 1 mm, the scale precision of the fixed-axis rotation external arc caliper and arc rack guide rail is set to 1°; the stroke of the support column is 80 mm˜120 mm, the first ball screw module is set with different strokes to adapt to standard operating tables of different widths, the strokes of the second ball screw module and third ball screw module are 80 mm˜120mm and 40˜80 mm respectively; the stroke of the arc rack guide rail is 110°˜140°, the self-rotation stroke of the self-rotating clamping assembly around the main pin axis is 220°˜270°.
